(19) United States
(12) Patent Application Publication
(10) Pub. No.: US 2005/0288371 A1
(43) Pub. Date: Dec. 29, 2005

(54) Treatment of neuropathic pain, fibromyalgia or rheumatoid arthritis

(75) Inventors: Bjarke Ebert, Farum (DK); Connie Sanchez, West Milford, NJ (US)

Correspondence Address: DARBY & DARBY P.C., P. O. BOX 5257, NEW YORK, NY, 10150-5257, US

(73) Assignee: H. Lundbeck A/S, Valby-Copenhagen (DK)

(21) Appl. No.: 11/020,630

(22) Filed: Dec. 22, 2004

Related U.S. Application Data
(63) Continuation-in-part of application No. PCT/DK04/00460, filed on Jun. 29, 2004.

(51) Int. Cl. A61K 31/195

(52) U.S. Cl. 514/554; 514/561

(57) ABSTRACT

The present invention relates to the use of gaboxadol for the preparation of medicaments useful for the treatment of neuropathic pain, fibromyalgia or rheumatoid arthritis.

This application is a continuation-in-part of International Application No. PCT/DK2004/000460 filed Jun. 29, 2004, which is incorporated herein by reference in its entirety.

### FIELD OF INVENTION

The present invention relates to the use of gaboxadol for the preparation of medicaments useful for the treatment of neuropathic pain, fibromyalgia or rheumatoid arthritis.

### BACKGROUND OF THE INVENTION

Neuropathic pain refers clinically to a group of chronic pain syndromes. They share the common feature that they are caused by an initial nerve damage, which subsequently results in abnormal sensory processing in the central and peripheral nervous system. Neuropathic pain conditions are the consequence of a number of diseases, e.g. diabetes, AIDS, multiple sclerosis, amputation and cancer. The available analgesic drugs often produce insufficient pain relief. Tricyclic antidepressants and some antiepileptic drugs, e.g. gabapentin, lamotrigine and carbamazepine are efficient in some patients. However, there is still a large unmet need for efficient drugs for the treatment of these conditions.

Most types of persistent, chronic non malignant pain, including fibromyalgia and arthritic pain, are characterized by an increased blood level of proinflammatory cytokines and a reciprocal relation between pain severity and sleep quality in that the pain interrupts sleep and this subsequently increase the pain sensation. These types of pain are characterized by prominent intrusion of alpha rhythms in the sleep EEG leading to an impaired slow wave sleep. Thus, if it were possible to counteract the effects mediated via the cytokines or, conversely, normalize the sleep pattern, it might be possible to obtain both improved analgesic activity and improved sleep quality and thus, the synergy between these two mechanism might be able to improve the quality of life for these particular groups of patients.

It has now, surprisingly, been found that gaboxadol (THIP) described in EP Patent 0840601 B1, herein incorporated by reference, in the formalin pain model and in a dose dependent manner, potently inhibited the pain response in phase 2. This second phase of the pain response, which is mediated via several proinflammatory mediators, is thought to mimic or lead to some of the key features of neuropathic pain, fibromyalgia, and rheumatoid arthritis, amongst others, and to prolonged pain and reduced sensitivity to the strong opioid analgesics.

### DESCRIPTION OF THE INVENTION

According to the present invention, a medicament for the treatment of neuropathic pain, fibromyalgia or rheumatoid arthritis is provided.

More specifically, the present invention relates to the use of gaboxadol having the general formula 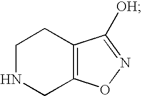
and throughout the description “gaboxadol” is intended to include any form of the compound, such as the free base (zwitter ion), pharmaceutically acceptable salts, e.g. pharmaceutically acceptable acid addition salts, hydrates or solvates of the base or salt, as well as anhydrates, and also amorphous, or crystalline forms.

In one embodiment, the present invention relates to the use of gaboxadol for the preparation of a medicament for the treatment of neuropathic pain.

In another embodiment, the present invention relates to the use of gaboxadol for the preparation of a medicament for the treatment of fibromyalgia.

In a further embodiment, the present invention relates to the use of gaboxadol for the preparation of a medicament for the treatment of rheumatoid arthritis.

In a further embodiment, gaboxadol is selected from the zwitter ion, typically a hydrate thereof, although the anhydrate is also suitable. A typical embodiment is the zwitter ion monohydrate.

In a further embodiment, gaboxadol is selected from an acid addition salt, typically a pharmaceutically acceptable acid addition salt. A typical embodiment is an organic acid addition salt, such as any one of the maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, p-amino-benzoic, glutamic, benzene sulfonic or theophylline acetic acid addition salts. Another typical embodiment is an inorganic acid addition salt, such as any one of the hydrochloric, hydrobromic, sulfuric, sulfamic, phosphoric or nitric acid addition salts.

Typically, gaboxadol is in the form of the hydrochloric acid salt, the hydrobromic acid salt, or the zwitter ion monohydrate.

In a further embodiment, gaboxadol is crystalline, such as the crystalline hydrochloric acid salt, the crystalline hydrobromic acid salt, or the crystalline zwitter ion monohydrate.

In a further aspect, the present invention relates to a method for the treatment of neuropathic pain, fibromyalgia or rheumatoid arthritis, comprising administering to a subject in need thereof an effective amount of gaboxadol and a pharmaceutically acceptable carrier.

Gaboxadol may be administered as an oral dose form, such as a solid oral dose form, typically tablets or capsules, or as a liquid oral dose form. Gaboxadol is most conveniently administered orally in unit dosage forms, such as tablets or capsules, containing the active ingredient in an amount from about 0.1 to about 150 mg/day, from about 0.2 to about 100 mg/day, from about 0.5 to about 50 mg/day, from about 1 to about 15 mg/day, or from about 2 to about 5 mg/day. Typically, the pharmaceutical composition comprises from 2.5 mg to 20 mg, such as from 5 mg to 15 mg of gaboxadol. The amount of gaboxadol is calculated based on the free base (zwitter ion) form.

The subject, such as a human, to be treated with gaboxadol may in fact be any subject of the human population, male or female, which may be divided into children, adults, or elderly. Any one of these patient groups relates to an embodiment of the invention. In one embodiment, the human subject does not suffer from a sleep disorder or sleep condition. In another embodiment, the human subject suffers from interruptions in sleep or other problems sleeping caused by neuropathic pain, fibromyalgia, or arthritis but does not suffer from any separate sleep disorder or sleep condition such as sleep apnea.

According to the invention, gaboxadol may be used as the base (the zwitter ion) or as a pharmaceutically acceptable acid addition salt thereof or as an anhydrate or hydrate of such salt or base. The salts of the compound used in the invention are salts formed with non-toxic organic or inorganic acids. Exemplary of such organic salts are those with maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, p-amino-benzoic, glutamic, benzene sulfonic and theophylline acetic acids, as well as the 8-halotheophyllines, for example 8-bromo-theophylline. Exemplary of such inorganic salts are those with hydrochloric, hydrobromic, sulfuric, sulfamic, phosphoric and nitric acids. Gaboxadol may also be used as the zwitter ion, e.g. the mono hydrate thereof.

The acid addition salts according to the invention may be obtained by treatment of gaboxadol with the acid in an inert solvent followed by precipitation, isolation and optionally re-crystallization by known methods and if desired micronization of the crystalline product by wet or dry milling or another convenient process, or preparation of particles from a solvent-emulsification process. Suitable methods are described in EP patent 0000338, for example.

Precipitation of the salt is typically carried out in an inert solvent, e.g. an inert polar solvent such as an alcohol (e.g. ethanol, 2-propanol and n-propanol), but water or mixtures of water and inert solvent may also be used.

According to the invention, gaboxadol or a pharmaceutically acceptable salt thereof may be administered in any suitable way, e.g. orally or parenterally, and it may be presented in any suitable form for such administration, e.g. in the form of tablets, capsules, powders, syrups or solutions or dispersions for injection. Preferably, and in accordance with the purpose of the present invention, gaboxadol is administered in the form of a solid pharmaceutical entity, suitably as a tablet or a capsule or in the form of a suspension, solution or dispersion for injection.

Methods for the preparation of solid or liquid pharmaceutical preparations are well known in the art. See e.g., Remington's Pharmaceutical Sciences, 20^{th} edition, Mack Publishing Company, 2000. Tablets may thus be prepared by mixing the active ingredients with an ordinary carrier, such as an adjuvant and/or diluent, and subsequently compressing the mixture in a convenient tabletting machine. Examples of adjuvants and/or diluents comprise: corn starch, lactose, talcum, magnesium stearate, gelatine, lactose, gums, and the like. Any other adjuvant or additive such as colourings, aroma, preservatives, etc. may also be used provided that they are compatible with the active ingredients. The pharmaceutical compositions of the invention thus typically comprise an effective amount of gaboxadol and a pharmaceutically acceptable carrier.

A suitable formulation of gaboxadol is described in WO 02/094225 filed May 17, 2002, published Nov. 28, 2002. Without limiting the invention in any way, it is intended that any one of the aspects or embodiments of this patent application is suitable for the medicaments or pharmaceutical compositions herein. For example, WO 02/094225 entitled “Granular Preparations of Gaboxadol” relates to a specific melt granulation which is particularly useful for formulation of an acid addition salt, but the present invention is in no way limited to such a formulation.

As used herein, the term “neuropathic pain” includes, but is not limited to, a group of chronic pain syndromes caused by an initial nerve damage, which subsequently results in abnormal sensory processing in the central and peripheral nervous system. According to one embodiment, the neuropathic pain is non-malignant in origin. Examples of neuropathic pain which can be treated by gaboxadol or a pharmaceutically acceptable salt thereof include, but are not limited to: thoracic outlet obstruction syndromes; compression and entrapment neuropathies such as ulnar nerve palsy, carpal tunnel syndrome, peroneal nerve palsy, radial nerve palsy; diabetic peripheral neuropathy and Guillain-Barre syndrome. Further examples include pain associated with or resulting from: trauma caused by injury or surgical operation; tumors; bony hyperostosis; casts; crutches; prolonged cramped postures; hemorrhage into a nerve; exposure to cold or radiation; collagen-vascular disorders; metabolic diseases such as diabetes; infectious diseases such as Lyme disease and HIV; toxins such as emetine, hexobarbital, barbital, chlorobutanol, sulfonamides, phenytoin, nitrofurantoin, the vinca alkaloids, heavy metals, carbon monoxide, triorthocresylphosphate, orthodinitrophenol, and other solvents and industrial poisons; autoimmune reactions; nutritional deficiency, and vitamin B deficiency in particular; and metabolic disorders such as hypothyroidism, porphyria, sarcoidosis, amyloidosis, uremia and diabetes.

As used herein, the term “fibromyalgia” is a chronic pain illness. Symptoms associated with fibromyalgia include, but are not limited to widespread musculoskeletal pain, aches, stiffness, soft tissue tenderness, fatigue, and problems sleeping. The symptoms of a fibromyalgia patient are varied and the intensities of the symptoms change over time. The onset of fibromyalgia can often be traced to an injury or physical or emotional trauma. Fibromyalgia patients may have abnormal levels of serotonin and often have tender points on their bodies. The fatigue and sleep-related problems of fibromyalgia patients are associated with specific abnormalities in the stage 4 deep sleep where fibromyalgia patients may have bursts of awake-like brain activity, limiting the time they spent in deep sleep.

As used herein, the term “rheumatoid arthritis” includes but is not limited to a systemic disease affecting the entire body. It is an autoimmune disease which may be characterized by the inflammation of the membrane lining the joint, which causes pain, stiffness, warmth, redness or swelling.

Pharmacological Tests

The formalin pain model is a well-established animal model of persistent somatic pain (Dubuisson, D. and Dennis, S. G. Pain 4, 1977, 161-174). It has been described as a model of clinical inflammatory pain (Tjølsen, A. and Hole, K, In: Dickenson, A. H. and Besson, J.-M. R., Editors, 1997, The Pharmacology of Pain, Springer-Verlag, Berlin, 1-20). Formalin injected subcutaneously in a hind paw produces initially a local stimulation of the nociceptors; this is referred to as phase 1. This is followed by inflammatory processes and nerve sensitization (phase 2). The latter phase models the neuropathic pain condition. A number of drugs are active in the model, e.g. morphine and in particular antiepileptic drugs (e.g. gabapentin, lamotrigine, carbamazepine) that have shown beneficial effects in clinical neuropathic states. However, these treatments are accompanied by troublesome side effects. The non-steroid antiinflammatory drugs (NSAIDs) that are used for treatment of inflammatory processes in tissue and have relatively weak effects on neuropathic pain conditions have weak activity in the formalin model.

Experimental Procedure

Fifty microliters of 2.5% formalin were administered subcutaneously (s.c.) into the plantar region of the right hind-paw. Following the injection, the animal was placed into an observation chamber, and its subsequent nociceptive behavior observed. A mirror behind the observation chamber allowed the experimenter an unobstructed view of the injected paw. Observation of the animal's behavior was made from 0-5 min (phase 1) and 20-30 min (phase 2) following formalin injection. The total time the animal spent licking, biting or shaking the injected paw was recorded. All animals (n=6-10) received a formalin injection and a s.c. injection of either vehicle or test drug 30 min or 2 h before the formalin test.

Results

The testing of gaboxadol in the formalin pain model showed that the compound potently inhibited the pain response in phase 2, in a dose dependent manner.

Gaboxadol also showed potent and dose dependent inhibition of the pain response in phase 1.

Throughout this application various references are cited; the contents of each cited reference is incorporated herein by reference in its entirety for all purposes.

1. A method for the preparation of a pharmaceutical composition for the treatment of neuropathic pain, fibromyalgia or rheumatoid arthritis in a subject, comprising: formulating an effective amount of gaboxadol for the treatment of neuropathic pain, fibromyalgia or rheumatoid arthritis, and a pharmaceutically acceptable carrier.

2. The method of claim 1, wherein the pharmaceutical composition is suitable for treating neuropathic pain.

3. The method of claim 1, wherein the pharmaceutical composition is suitable for treating fibromyalgia.

4. The method of claim 1, wherein the pharmaceutical composition is suitable for treating rheumatoid arthritis.

5. The method of claim 1, wherein gaboxadol is in the form of an acid addition salt, a zwitter ion hydrate, or a zwitter ion anhydrate.

6. The method of claim 5, wherein the acid addition salt is a hydrochloride salt or a hydrobromide salt.

7. The method of claim 5, wherein the zwitter ion hydrate is a zwitter ion monohydrate.

8. The method of claim 1, wherein the pharmaceutical composition is an oral dose form.

9. The method of claim 8, wherein the oral dose form is a solid oral dose form or a liquid oral dose form.

10. The method of claim 9, wherein the solid oral dose form is a tablet or a capsule.

11. The method of claim 1, wherein gaboxadol is crystalline.

12. The method of claim 1, wherein the subject is a human.

13. The method of claim 1, wherein the effective amount of gaboxadol is from about 0.1 mg/day to about 50 mg/day.

14. A method for the treatment of neuropathic pain, fibromyalgia or rheumatoid arthritis in a subject, comprising: administering an effective amount of gaboxadol for the treatment of neuropathic pain, fibromyalgia or rheumatoid arthritis, and a pharmaceutically acceptable carrier.

15. The method of claim 14, wherein the effective amount of gaboxadol is effective for treating neuropathic pain.

16. The method of claim 14, wherein the effective amount of gaboxadol is effective for treating fibromyalgia.

17. The method of claim 14, wherein the effective amount of gaboxadol is effective for treating rheumatoid arthritis.

18. The method of claim 14, wherein the effective amount of gaboxadol is from about 0.1 mg/day to about 50 mg/day.

19. The method of claim 14, wherein the subject is a human.

20. A pharmaceutical composition for the treatment of neuropathic pain, fibromyalgia or rheumatoid arthritis in a subject comprising: an effective amount of gaboxadol for the treatment of neuropathic pain, fibromyalgia or rheumatoid arthritis, and a pharmaceutically acceptable carrier.
